(19) United States
(12) Patent Application Publication
(10) Pub. No.: US 2017/0218354 A1
(43) Pub. Date: Aug. 3, 2017

(54) HIGHLY POTENT CELLULOLYTIC ENZYME PREPARATIONS AND PROCESSES FOR PRODUCING SAME

(71) Applicant: DESIGNER ENERGY LTD., Rehovot (IL)

(72) Inventors: Ely MORAG, Rehovot (IL); Tal BARAK, Gedera (IL); Alon KARPOL, Tel-Mond (IL); Michael ANBAR, Tel Aviv (IL)

(73) Assignee: DESIGNER ENERGY LTD., Rehovot (IL)

(21) Appl. No.: 15/484,225

(22) Filed: Apr. 11, 2017

Related U.S. Application Data
(62) Division of application No. 14/759,564, filed on Jul. 7, 2015, now abandoned.
(60) Provisional application No. 61/750,827, filed on Jan. 10, 2013.

(51) Int. Cl. C12N 11/16; C12P 19/02; C12N 9/42; C12P 19/14

(52) U.S. Cl. CPC C12N 11/16; C12P 19/14; C12P 19/02; C12N 9/2445; C12Y 302/01021

(57) ABSTRACT

Compositions comprising unprocessed cell pellets of a cellulosome-producing microorganism grown on cellulosic biomass are provided. Further provided are methods for producing the compositions and uses thereof in hydrolysis of cellulosic substrates. In particular, the compositions advantageously contain extracellular beta-glucosidase, either expressed on the cells themselves or extrinsically added to the cell pellets.

### CROSS-REFERENCE TO RELATED APPLICATIONS

This application is a Divisional of U.S. patent application Ser. No. 14/759,564, which is the National Stage of International Patent Application No. PCT/IL2014/050024, filed Jan. 9, 2014, which claims the benefit of priority of U.S. Provisional Application No. 61/750,827, filed Jan. 10, 2013. The disclosures of each of application Ser. No. 14/759,564 and PCT/IL2014/050024 are expressly incorporated by reference herein in their entireties.

### FIELD OF THE INVENTION

The present invention relates to enzyme preparations for use in the degradation of cellulosic biomass. The present invention further relates to processes for producing the enzyme preparations and methods of degrading cellulosic substrates utilizing same.

### BACKGROUND OF THE INVENTION

Plant cell wall components, primarily cellulose and hemicellulose, offer an excellent source of carbon and energy. Producing ethanol from plant materials, for example, requires three consecutive steps: physiochemical pretreatments, hydrolysis of cellulose and hemicelluloses into soluble sugars, and fermentation into ethanol. The most challenging, time consuming and costly process is the hydrolysis step, and much effort has been given to reduce the cost of the treatment and make it efficient and cost effective. Lignocellulose, a matrix of cellulose, lignin and optionally hemicellulose, is also difficult to hydrolyze due to the high association of the cellulose with hemicellulose and surrounding lignin, which results in a highly ordered, tightly packed, crystalline structure.

Only a few microorganisms have acquired the capacity to degrade these structures into soluble sugars. For example, the thermophilic anaerobic bacterium Clostridium thermocellum exhibits one of the most highly efficient degradation systems termed “cellulosome”. The cellulosome is a multienzyme system thought to allow synergistic enzyme activity in close proximity to the bacterial cell. Cellulosomes are protuberances produced on the cell wall of cellulolytic bacteria when growing on cellulosic materials. These protuberances are stable enzyme complexes that are firmly bound to the bacterial cell wall but flexible enough to also bind tightly to microcrystalline cellulose. The cellulosome is comprised of a diversity of hydrolytic activities. Among them are endoglucanases that catalyze the hydrolysis of internal bonds inside the cellulose chain and randomly produce new chain ends; and exoglucanases that cleave the cellulose chain at the exposed ends to produce mainly cellobiose. Other enzymes include xylanases, carbohydrate esterases, pectin lyases and others.

Cellulosomal protein complexes are routinely used in two main ways. The first is as part of an enzyme-microbe assembly of live and growing bacteria (see, for example, Lu et al., 2006, Enzyme-microbe synergy during cellulose hydrolysis by Clostridium thermocellum. Proc Natl Acad Sci USA 103:16165-9). In this method the bacteria are typically grown at the optimal conditions for their growth on lignocellulosic material, and the soluble sugars produced form the cell-associated and detached cellulases are used for yeast fermentation or are directly converted into ethanol by the bacteria. The second method involves extraction and isolation of sheared cellulosomal complexes in the bacterial medium during stationary phase of growth. The isolated cellulosomal complexes are then used for the degradation of cellulosic material. Both these methods have several limitations, for example, in the amount of biomass loading, bacteria:biomass ratios and high sensitivity to inhibitors.

Most of the research on cellulosomes was done on cell-free cellulosomal components. Very little is known about the potential activity of cell-bound cellulosomes produced with natural substrates, as the work that has been published so far has been limited to cells growing on Cellobiose or Avicel (Zhang et al., 2003. Quantification of cell and cellulase mass concentrations during anaerobic cellulose fermentation: development of an enzyme-linked immunosorbent assay-based method with application to Clostridium thermocellum batch cultures. Anal Chem 75:219-27).

Nowhere is it disclosed or suggested that the pellet of a cellulosome-producing microorganism grown on a cellulosic biomass can be collected and used for cellulose degradation on a commercial scale, without processing or purification of the enzymatic or cellular components.

There still remains a need for improved degradation of biomass, especially recalcitrant cellulosic biomass. For example, it would be highly beneficial to have compositions for cellulose degradation that are simple and economic to produce while showing high cellulolytic activity.

### SUMMARY OF THE INVENTION

The present invention provides compositions comprising pellets of cellulosome-producing microorganisms, processes for producing them and methods for degrading cellulosic substances using the same.

The present invention discloses for the first time that crude pellet preparations of a cellulosome-producing microorganism grown on a cellulosic biomass can be collected and used for efficient degradation of cellulosic substrates without further processing. The present invention is based in part on the finding that the pellet fraction collected following growth of cellulosome-producing bacteria in the presence of cellulosic biomass show a remarkable cellulolytic activity. In contrast to hitherto described methods that utilize cell-free cellulosomes and cellulosome components to hydrolyze cellulosic materials, the pellet is used as a crude preparation without any purification steps to isolate enzymes or separate the cells and other materials contained within the pelleted fraction, such as residual un-hydrolyzed biomass. Surprisingly, the activity of the crude pellet was found to be superior to that of known commercial enzyme cocktails, as exemplified hereinbelow.

Advantageously, the cells of the cellulosome-producing microorganism contained within the pellet are inactivated according to the principles of the present invention, thereby permitting a wider range of conditions for cellulose hydrolysis compared to cellulose hydrolysis by living cells. In addition, as the microorganism is non-vital, no assimilation of soluble sugars occurs during the hydrolysis, and higher concentration of soluble sugars can be achieved.

The compositions of the present invention further comprise a beta-glucosidase. The addition of a beta-glucosidase to a preparation of unprocessed pellet according to embodiments of the present invention was surprisingly found to significantly improve the activity of the preparation and resulted in enhanced cellulose degradation, as exemplified herein below.

According to one aspect, the present invention provides a cellulolytic enzyme composition comprising an unprocessed pellet of a cellulosome-producing microorganism, wherein cells of the cellulosome-producing microorganism contained within the pellet are inactivated; and further comprising detectable extra-cellular beta-glucosidase.

In some embodiments, the pellet is characterized by a protein:cellulosic material ratio in the range of about 6:1-1:10 (w/w), for example about 3:1-1:5 (w/w), and a DNA:cellulosic material ratio in the range of about 1:800-1:50 (w/w), for example about 1:400-1:25 (w/w).

As used herein, “pellet” refers to the insoluble components of the medium following culturing. The term “supernatant” encompasses the soluble components of the medium following culturing. Advantageously, according to embodiments of the present invention, the microorganisms are grown on cellulosic material. Following culturing, the supernatant typically contains free enzymes, cellulosome complexes detached from cells, soluble sugars released during the degradation of the cellulosic material used for growing the microorganism, nutrients and the like. The pellet generally comprises inactivated cells of the cellulosome-producing microorganism, cell-bound cellulosome complexes, and residual un-hydrolyzed or partially hydrolyzed cellulosic material with cellulolytic enzymes bound thereto. The compositions of the present invention comprise the pellet components and are substantially devoid of the supernatant components. As used herein, “substantially devoid of supernatant components” indicates that the compositions comprise less than 10%, preferably less than 5%, less than 3%, less than 1%, less than 0.5% (w/w) of residual supernatant components.

The microorganism cells in the composition of the present invention are inactivated. As used herein, “inactivated”, indicates dead or dying cells. Typically, at least 80% of the cells are inactivated, for example at least 85% of the cells are inactivated, at least 90% of the cells are inactivated, at least 95% of the cells are inactivated, or 100% of the cells are inactivated. Each possibility represents a separate embodiment of the invention.

The protein:cellulosic material ratio of the pellet refers to the weight ratio between the total amount of proteins and the total amount of residual cellulosic material that was added to the culture medium of the cellulosome-producing microorganism but was not hydrolyzed. The DNA:cellulosic material ratio of the pellet refers to the weight ratio between the total amount of DNA and the total amount of residual un-hydrolyzed cellulosic material.

In some embodiments, the cellulosic material added to the culture medium is purified cellulose. According to these embodiments, the protein:cellulosic material ratio refers to the ratio between the total amount of protein to the total amount of cellulose in the pellet. Similarly, the DNA:cellulosic material ratio refers, according to these embodiments, to the ratio between the total amount of DNA to the total amount of cellulose in the pellet.

In other embodiments, the cellulosic material is a ligno-cellulosic material containing cellulose, hemicellulose and lignin. According to these embodiments, the protein:cellulosic material ratio refers to the ratio between the total amount of protein and the total amount of cellulose, hemicellulose and lignin in the pellet. Similarly, the DNA:cellulosic material ratio refers, according to these embodiments, to the ratio between the total amount of DNA to the total amount of cellulose, hemicellulose and lignin in the pellet.

As used herein the term “unprocessed pellet” may be used interchangeably with the term “crude pellet” and refers to a pellet preparation that has not been subjected to purification processes to isolate enzymes or separate cells. According to further embodiments, the crude pellet is not treated to remove residual unhydrolyzed biomass or other components. The pellet may be subjected to other processing procedures, such as drying as a non-limiting example.

In some embodiments, the pellet is a wet pellet. In other embodiments, the pellet is dried to provide a dry composition. In some embodiments, the dried pellet comprises about 3-20% residual water.

In some embodiments, the cellulosome-producing microorganism is anaerobic. In other embodiments, the cellulosome-producing microorganism is aerobic.

In some embodiments, the cellulosome-producing microorganism is thermophilic. In other embodiments, the cellulosome-producing microorganism is mesophilic.

In some embodiments, the cellulosome-producing microorganism is a bacterium.

In some embodiments, the bacterium is an anaerobic thermophilic bacterium. In some embodiments, the anaerobic thermophilic bacterium is selected from the group consisting of Clostridium thermocellum, Clostridium clariflavum and Clostridium josui. Each possibility represents a separate embodiment of the invention.

In some embodiments, the bacterium is an anaerobic mesophilic bacterium. In some embodiments, the anaerobic thermophilic bacterium is selected from the group consisting of Clostridium cellulolyticum, Clostridium cellulovorans, Acetivibrio cellulolyticus, Bacteroides cellulosolvens and a Ruminococcus species. Each possibility represents a separate embodiment of the invention.

In some embodiments, the cellulosome-producing microorganism is a fungus.

In some embodiments, the fungus is an anaerobic, mesophilic fungus. In some embodiments, the anaerobic, mesophilic fungus is selected from the group consisting of a Neocallimastix species, a Piromyces species and an Orpinomyces species. Each possibility represents a separate embodiment of the invention.

In some embodiments, the beta-glucosidase in the composition is recombinantly produced by the cellulosome-producing microorganism. According to these embodiments, the microorganism is genetically engineered to produce the beta-glucosidase as a recombinant enzyme during its growth in the culture. The beta-glucosidase according to these embodiments is secreted, extra-cellular and it is possible to detect its activity after collection of the pellet. The beta-glucosidase according to these embodiments is typically incorporated into the cellulosome of the microorganism, for example via addition of a dockerin module that interacts with a cohesin module of a scaffoldin subunit of the cellulosome, and therefore remains in the pellet after its collection. Alternatively, the beta-glucosidase has a cellulose-binding module that actively hinds the enzyme to the substrate in the pellet.

In other embodiments, the beta-glucosidase is exogenously added to the composition. As used herein, the phrase “exogenously added”, when referring to the beta-glucosidase, indicates that an isolated beta-glucosidase is added to the composition, rather than produced by the microorganism in the culture. Typically, the beta-glucosidase is added after collection of the pellet. Isolated beta-glucosidases are commercially available and may also be produced recombinantly. In some embodiments, the exogenously added beta-glucosidase includes a dockerin or a cohesin module and can incorporate into the cellulosome complexes present in the composition. It is to be understood that in order for the beta-glucosidase to incorporate into cellulosome complexes present in the composition it should include a dockerin module matching a cohesin module of the cellulosomal scaffoldin subunits in the composition, or a cohesin module (for example a type-II cohesin) matching a dockerin module of the cellulosomal scaffoldin subunits. In additional embodiments, the exogenously added beta-glucosidase includes a cellulose-binding module (CBM) for substrate binding. In other embodiments, the exogenously added beta-glucosidase does not include a dockerin, cohesin or CBM and acts as a free enzyme in the composition.

In some embodiments, the exogenously added beta-glucosidase is a thermostable beta-glucosidase, which is thermostable at temperatures above about 50° C. As used herein, “thermostable” at a given temperature means that the enzyme retains its activity when the enzymatic reaction is performed in that temperature.

In some embodiments, the exogenously added beta-glucosidase has an optimum temperature for activity of about 50° C. or higher. As used herein, an “optimum temperature for activity” refers to the temperature for an enzyme at which it shows maximum activity.

In some embodiments, the beta-glucosidase is thermostable at temperatures above about 60° C., for example above about 70° C., in the range of about 60-80° C. Each possibility represents a separate embodiment of the invention.

In some embodiments, the beta-glucosidase has an optimum temperature for activity of about 60° C. or higher, about 70° C. or higher, in the range of about 60-80° C. Each possibility represents a separate embodiment of the invention.

In some embodiments, the beta-glucosidase originates from a thermophilic microorganism.

In some embodiments, the amount of the exogenously added beta-glucosidase in the composition is in the range of about 0.01-10% (w/w), for example 0.01-5% (w/w). Each possibility represents a separate embodiment of the invention.

According to another aspect, the present invention provides a process for producing a cellulolytic enzyme preparation, the process comprising: (i) culturing a cellulosome-producing microorganism in a medium comprising a cellulosic material until late exponential phase or stationery phase, (ii) centrifuging or filtering the medium following said culturing to obtain a pellet; (iii) separating the obtained pellet from the supernatant and inactivating the cells of the microorganism; and (iv) using the obtained pellet without purification of enzymes or other components to enzymatically hydrolyze cellulosic substrates.

In some embodiments, the cellulosic material is purified cellulose. In some embodiments, the cellulosic material is microcrystalline cellulose.

In other embodiments, the cellulosic material is a complex cellulosic material. As used herein, the term “complex cellulosic material” refers to a cellulosic substrate which comprises cellulose and additional one or more complex polysaccharides and/or other polymers, typically polysaccharides and polymers of plant cell walls, such as hemicellulose and lignin. In some embodiments, the cellulosic material is a ligno-cellulosic material.

In some embodiments, the ligno-cellulosic material is selected from the group consisting of wheat straw, switchgrass, corn cob, corn stover, sorghum straw, cotton straw, bagasse, energy cane, hard wood paper, soft wood paper, and a combination thereof. Each possibility represents a separate embodiment of the invention.

In some embodiments, the ligno-cellulosic material is derived from a plant selected from the group consisting of a Populous species, a Salix species, an Acacia species, a Tamarix species, Arundo donax, Miscanthus giganteus, and a combination thereof. Each possibility represents a separate embodiment of the invention.

In some embodiments, the cellulosic material is a pre-treated cellulosic material. In some embodiments, the pretreatment comprises chemical pretreatment, physical pretreatment or a combination thereof. Each possibility represents a separate embodiment of the invention.

In some embodiments, the medium contains about 0.1-25% (w/v) cellulosic material.

In some embodiments, the medium further comprises one or more plant polysaccharides. In some embodiments, the medium further comprises pectin.

In some embodiments, the process is a batch process. In some embodiments, the microorganism is cultured for about 12-100 hours.

It is to be understood that the step of inactivating the cells of the microorganism may precede the step of centrifuging or filtering the medium following culturing, or the step of separating the obtained pellet from the supernatant. Typically, at least 80% of the cells are inactivated following the inactivating step, for example at least 85% of the cells are inactivated, at least 90% of the cells are inactivated, at least 95% of the cells are inactivated, or 100% of the cells are inactivated following the inactivating step.

In some embodiments, the process further comprises adding a beta-glucosidase to the pellet preparation.

According to yet another aspect, the present invention provides a method for degrading a cellulosic substrate, the method comprising contacting the cellulosic substrate with the composition of the present invention.

According to yet another aspect, the present invention provides a method for degrading a cellulosic substrate, the method comprising obtaining the cellulolytic enzyme preparation of the present invention, and contacting the cellulosic substrate with said cellulolytic enzyme preparation.

In some embodiments, the cellulosic substrate is purified cellulose. In other embodiments, the cellulosic substrate comprises cellulose and hemicelluloses. In additional embodiments, the cellulosic substrate comprises cellulose, hemicelluloses and lignin.

In some embodiments, the degradation methods further comprise recycling the enzyme composition for one or more successive hydrolyses. In some embodiments, recycling comprises contacting a first sample of a cellulosic substrate with the enzyme composition to obtain hydrolysis of the first sample of a cellulosic substrate into hydrolysis products; (ii) collecting the hydrolysis products; and (iii) contacting a second sample of a cellulosic substrate with the enzyme composition to obtain hydrolysis of the second sample of a cellulosic substrate into hydrolysis products.

In some exemplary embodiments, collecting the hydrolysis products is performed by centrifuging the reaction mixture to obtain a sediment comprising the enzyme composition and a supernatant comprising the hydrolysis products, and collecting the supernatant.

These and further aspects and features of the present invention will become apparent from the figures, detailed description, examples and claims which follow.

### BRIEF DESCRIPTION OF THE FIGURES

FIG. 1. Production of cell-associated cellulosomal pellet by Clostridium thermocellum (Ct) and Clostridium clariflavum (Cc).

FIG. 2. Production of cell-associated cellulosomal pellet on different biomass types. C. thermocellum was grown for a period of 24-48 hours at 60° C. in anaerobic conditions. The wet-weight was determined upon centrifugation and complete biomass digestion.

FIG. 3. Hydrolysis of 10% MCC after 20 hours at 70° C. with cell-associated cellulosomal pellet produced using different biomass. The amount of soluble sugars was determined using DNS method.

FIG. 4. Example of two compositions of cell-associated cellulosomal pellet using two types of biomass and pretreatments. Black bars represent protein concentration and white bars represent biomass concentration determined by total reducing sugar content after complete digestion.

FIG. 5. Hydrolysis of 10% MCC by cell-associated cellulosomal pellet (with beta glucosidase) and commercial enzyme cocktail (Accelerase 1500) in 10% w/w enzyme/biomass ratios at 70° C. and 50° C. respectively. Black bars represent cell-associated cellulosomal pellet, white bars represents a commercial cocktail.

FIG. 6. Activity on 10% MCC of cell-associated cellulosomal pellet (10% w/w) and beta-glucosidase at 60° C. (black bars) and 70° C. (white bars).

FIG. 7. Influence of beta glucosidase on hydrolysis of 10% MCC by cell-associated cellulosomal pellet at 70° C. Black bars represent cell-associated cellulosomal pellet, white bars represents cell-associated cellulosomal pellet with addition of beta glucosidase at a 0.032% w/w.

FIG. 8. Consecutive hydrolysis of pretreated biomass. 3 rounds of enzyme recycling were done for 24 hours each at 70° C.

### DETAILED DESCRIPTION OF THE INVENTION

The present invention is directed to compositions for use in the degradation of cellulosic substrates that are simple and economic to produce while being highly potent, and processes for producing them. The present invention is further directed to cellulose hydrolysis using said compositions.

The principles of the present invention encompass a number of advantages, for example:

1. Simple production that does not require recombinant expression and purification of enzymes, or time consuming filtration or precipitation of cellulosome from medium.

2. The pellet preparation is very potent and shows very high activity levels.

3. As opposed to direct anaerobic fermentation there is no limitation to cellulose concentration that is used in the hydrolysis and aerobic conditions could be used.

4. The pellet preparation is robust and hydrolysis could be done in a wider range of conditions compared to hydrolysis using live, growing bacteria.

5. As the microorganism is inactivated, no assimilation of soluble sugars occurs during the hydrolysis, and higher concentration of soluble sugars can be achieved. As exemplified herein below, an enzymatic preparation prepared from a culture of an anaerobic bacterium remained highly active even though the bacterial cells have been exposed to aerobic conditions and were therefore non-vital.

6. Higher hydrolysis temperatures can be used when the preparation is made from thermophilic microorganisms, which enhance the hydrolysis rate, decrease contaminations and improve mass transfer rates.

Microorganism Growth:

Microorganisms:

As used herein, the term “cellulosome-producing microorganisms” refers to microorganisms (including bacteria and fungi) with cellulose utilizability that produce cellulosome complexes. The cellulosome is a multi-enzyme complex of cellulases, hemicellulases and other carbohydrate-active enzymes. Its basic structure typically contains a non-catalytic subunit called scaffoldin that binds the insoluble cellulosic substrate via a cellulose-specific carbohydrate-binding module (CBM). The scaffoldin subunit typically contains a set of subunit-binding modules, termed cohesins, that mediate specific incorporation and organization of the various enzymatic subunits into the complex through a complementary binding module, termed dockerin, that is present in each enzymatic subunit. The assembly of the enzymes into the complex ensures their collective targeting to a specific region of the substrate thereby facilitating stronger synergism among the catalytic components. The cellulosomes are typically bound to the cell surface of the microorganism but may also be secreted to the surrounding environment. For example, some cellulosome-producing bacteria release their cell-bound cellulosomes during the stationary growth phase. In addition to cellulosome complexes, a cellulosome-producing microorganism may also produce free, non-cellulosomal enzymes that degrade cellulosic substances.

The cellulosome-producing microorganisms utilized herein may include aerobic and anaerobic, thermophilic and mesophilic microorganisms. Each possibility represents a separate embodiment of the invention.

In some embodiments, the cellulosome-producing microorganism is a bacterium.

Examples of suitable bacteria include, but are not limited to, Clostridium thermocellum (anaerobic, thermophilic), Clostridium clariflavum (anaerobic, moderate thermophilic), Clostridium cellulolyticum (anaerobic, mesophilic), Clostridium cellulovorans (anaerobic, mesophilic), Clostridium josui (anaerobic, moderate thermophilic), Acetivibrio cellulolyticus (anaerobic, mesophilic), Bacteroides cellulosolvens (anaerobic, mesophilic) and a Ruminococcus species (anaerobic, mesophilic). Each possibility represents a separate embodiment of the invention.

In some embodiments, the cellulosome-producing microorganism is a fungus.

Examples of suitable fungi include, but are not limited to, a Neocallimastix species (anaerobic, mesophilic), a Piromyces species (anaerobic, mesophilic) and an Orpinomyces species (anaerobic, mesophilic). Each possibility represents a separate embodiment of the invention.

Biomass:

According to the principles of the present invention, the cellulosome-producing microorganisms are grown in the presence of a cellulosic material. As used herein, the terms “cellulosic materials” and “cellulosic biomass” are used interchangeably and refer to materials that contain cellulose, in particular materials derived from plant sources that contain cellulose. Cellulose is a linear polysaccharide polymer composed of β-1,4 linked D-glucose molecules. It is the major component of plant cell walls.

In some embodiments, the cellulosic material is purified cellulose. In some embodiments, the cellulosic material is microcrystalline cellulose. As used herein, “microcrystalline cellulose” refers to purified, partially depolymerized cellulose prepared by treating alpha cellulose. Examples of microcrystalline cellulose include microcrystalline cellulose sold under the trade name Avicel®.

In other embodiments, the cellulosic material is a complex cellulosic material which comprises cellulose and additional one or more complex polysaccharides or other polymers, typically polysaccharides and polymers of plant cell walls, such as hemicellulose and lignin.

As used herein, the term “hemicellulose” has its art-known meaning and refers to a group of branched polysaccharide hetero- or homo-polymers composed of a variety of sugar monomers. Hemicellulose includes, for example, xylan, glucuronoxylan, arabinoxylan, glucomannan, and xyloglucan, in complex branched structures with a spectrum of substituents.

As used herein, the term “lignin” has its art-known meaning and refers to a polymeric material, mainly composed of linked phenolic monomeric compounds, such as p-coumaryl alcohol, coniferyl alcohol, and sinapyl alcohol, which forms the basis of structural rigidity in plants and is frequently referred to as the woody portion of plants. Lignin is also considered to be the non-carbohydrate portion of the cell wall of plants.

In some embodiments, the cellulosic material comprises cellulose, hemicellulose, and lignin. A cellulosic material containing cellulose, hemicellulose and lignin may be referred to as a “ligno-cellulosic material”. In some embodiments, the cellulosic material is ligno-cellulosic.

The cellulosic material may include natural plant biomass and also paper waste and the like. Examples of suitable cellulosic materials include, but are not limited to, wheat straw, switchgrass, corn cob, corn stover, sorghum straw, cotton straw, bagasse, energy cane, hard wood paper, soft wood paper, and combinations thereof. Each possibility represents a separate embodiment of the invention.

Additional examples include plant biomass of a Populous species, a Salix species, an Acacia species, a Tamarix species, Arundo donax, Miscanthus giganteus and combinations thereof. Each possibility represents a separate embodiment of the invention. The plant biomass may be derived from stems, leaves, hulls and husks.

Pre-Treatment:

In some embodiments, the cellulosic material is pre-treated prior to its utilization for the microorganisms growth, to increase its susceptibility to hydrolysis. Pretreatment may include chemical and physical pretreatments or combinations thereof, and can be performed by methods known in the art. Exemplary pretreatment procedures are provided in the Examples section below.

Physical pre-treatment techniques include, for example, various types of milling/comminution (reduction of particle size), irradiation, steaming/steam explosion, and hydrothermolysis.

Chemical pre-treatment techniques include, for example, acid, dilute acid, base, organic solvent, lime, ammonia, sulfur dioxide, carbon dioxide, pH-controlled hydrothermolysis, wet oxidation, and solvent treatment.

Acid pre-treatments are typically performed using sulfuric acid. Other acids may be used, such as hydrochloric acid, phosphoric acid, nitric acid or any mixture thereof. In general, the acid is usually mixed or contacted with the cellulosic material and the mixture is held at a temperature in the range of about 25-180° C. for a period ranging from about 1-60 minutes.

Alkali pre-treatments may be performed using sodium hydroxide, ammonia, calcium hydroxide and potassium hydroxide. In general, the base is usually mixed or contacted with the cellulosic material and the mixture is held at a temperature in the range of about 0-130° C. for a period ranging from about 5-300 minutes.

A particular example for a chemical pretreatment includes mercerization. An exemplary procedure is provided below.

Composition of the Growth Medium:

The above described cellulosic materials are added to the growth medium of the microorganisms. The culture medium is typically an aqueous medium. In some embodiments, the medium contains about 0.1-25% cellulosic material or any amount therebetween, for example about 0.5-15% or any amount therebetween, about 0.5-5% or any amount therebetween. Each possibility represents a separate embodiment of the invention. As defined herein, the concentration of the cellulosic material in the medium is a ratio by weight of dry cellulosic material to a culture medium volume.

As used herein, the term “about”, when referring to a measurable value such as an amount, a temporal duration, and the like, is meant to encompass variations of +/−10%, more preferably +/−5%, even more preferably, +/−1%, and still more preferably +/−0.1% from the specified value, as such variations are appropriate to perform the disclosed methods.

In some embodiments, the culture medium further comprises one or more complex plant polysaccharides, such as pectin. Suitable amounts of pectin are in the range of about 0.5-10 g/l.

The culture medium typically further comprises nitrogen and phosphate sources, and may also include appropriate salts, minerals, metals and other nutrients as known to a person of skill in the art.

Nitrogen sources may include, for example, ammonia, such as ammonium sulfate, and peptides. A nutrient source containing protein hydrolysate and amino acids such as yeast extract and peptone may also be used.

Examples of minerals include minerals and mineral salts that can supply nutrients comprising P, K, Mg, S, Ca, Fe, Zn, Mn, and Cu.

Further, for suppressing pH decrease due to the proliferation, a buffering agent may be added.

In the case of anaerobic fermentation, a gas phase of the culture medium may be substituted by carbon dioxide gas or nitrogen gas.

The above media components may be added prior to, simultaneously with or shortly after inoculation of the culture with the microorganism.

Culture Conditions:

Culture conditions may be set up according to optimum temperature and optimum pH of the particular microorganism used. In addition, culture is performed under aerobic or anaerobic conditions, according to the nature of the microorganism used. The optimal pH and temperature for each microorganism, as well as information regarding the microorganism being aerobic or anaerobic, can be readily found in the scientific literature, for example, publications and information available from the DSMZ (Deutsche Sammlung von Mikroorganismen and Zellkulturen) GmbH, Braunschweig Germany.

The culturing process according to the present invention is typically a batch process. In a batch process, all the necessary materials, with the exception of oxygen for aerobic processes, are placed in a reactor at the start of the operation and the fermentation is allowed to proceed until completion, at which point the product is harvested. The microorganisms can be cultured in conventional fermentation bioreactors. A starter culture can be grown prior to culturing in bioreactors, as known in the art. The obtained started culture is than inoculated to a bioreactor.

The microorganisms are typically grown until late exponential phase or stationery phase, for example early stationery phase (e.g. about 12-24 hr), or late stationery phase (e.g., about 24 hours after the stationery phase begins).

In some embodiments, the microorganism are cultured for a period of time ranging from about 12-100 hours or any amount therebetween, for example from about 24-72 hours, from about 24-48 hours or any amount therebetween.

The exact duration of incubation can be determined by one or more of the following:

NaOH consumption—NaOH is added to the culture medium in order to neutralize an acid formed during growth of the microorganisms. Fermentation is stopped and the pellet is collected when NaOH is no longer consumed, or about 24 hours after NaOH is no longer consumed.

Preliminary assay—a preliminary assay can be performed in order to determine the incubation duration in which the highest level of cellulolytic activity is obtained from the collected pellet. The preliminary assay includes the following steps: (i) culturing a cellulosome-producing bacteria of choice in a medium containing a cellulosic substrate of choice; (ii) sampling the culture at various time points: for example, samples may be taken out every 6-10 hours, or at particular time points, such as 12 hours, 18 hours, 24 hours, 36 hours, 60 hours and 72 hours after the beginning of incubation; (iii) testing each sample for its cellulolytic activity (for example, on microcrystalline cellulose); (iv) identifying the sample with the maximum cellulolytic activity, and accordingly the time point in which maximum activity is obtained. This time point would be the optimal incubation duration for the particular microorganism in the particular medium. This incubation duration should be used for preparing the enzymatic compositions of the present invention.

Cellulolytic Enzyme Preparation:

Following growth, the pellet components of the culture broth are separated from supernatant components. The separation may be performed by any known method, including centrifugation and filtration through a micro-sieve membrane.

In some embodiments, the culture broth is subjected to centrifugation to obtain a pellet. Typically, centrifugation is performed for about 10-20 minutes, at about 3200-17,000 g. Following centrifugation, the pellet is collected, namely, separated from the supernatant. Separation of the pellet from the supernatant may be performed by any known method, for example by pouring off or filtering the supernatant.

Pellet Composition and Characterization

The collected pellet fraction comprises insoluble components that were present in the culture broth. These components include cells and/or cell debris, cell-bound cellulosomes, residual un-hydrolyzed or partially hydrolyzed cellulosic biomass, and biomass-bound cellulolytic enzymes.

The cells present in the pellet are inactivated according to the principles of the present invention. In some embodiments, when anaerobic microorganisms are used, they are inactivated upon exposure to oxygen. Thus, inactivating anaerobic microorganism typically occurs during the collection and processing of the culture broth to obtain a pellet. For aerobic microorganisms, inactivation may be performed, for example, by exposing the cells to an azide, such as sodium azide. In some embodiments, inactivation of aerobic microorganisms is performed by a method other than sonication. In some embodiments, inactivation of the microorganisms is performed without affecting cell integrity. Typically, at least 80% of the cells are inactivated, for example at least 85%, at least 90%, at least 95%, or 100% of the cells are inactivated.

According to the principles of the present invention, the compositions comprise an unprocessed pellet that has not been subjected to purification processes to isolate or remove particular components from the pellet, including for example various chromatography procedures to isolate enzymatic components, or sonication and centrifugation to separate and remove cells from the composition.

In some embodiments, the pellet is subjected to processing other than purification of enzymes or other components of the pellet.

The pellet may be used as a wet preparation, or can be dried by methods known in the art such as spray-drying or lyophilization.

Thus, in some embodiments, the compositions of the present invention comprise a wet pellet of a cellulosome-producing microorganism. In some embodiments, the wet pellet is characterized by water content in the range of 60-80% or any amount therebetween, for example about 65-75% water or any amount therebetween. Each possibility represents a separate embodiment of the invention. Determination of the water content may be performed by any method known to a person of skill in the art, such as determining mass loss following heating or oven drying.

In other embodiments, the compositions of the present invention comprise a dried pellet of a cellulosome-producing microorganism. In some embodiments, the dried pellet comprises about 3-20% residual water, for example about 3-10%, or about 3-5% residual water.

The pellet (wet or dry) may be characterized by determining one or more of the following:

Protein content: may be determined, for example, using colorimetric methods, such as the bicinchoninic acid (BCA) protein assay. The protein content is calculated using a calibration curve of a known protein (e.g. bovine serum albumin).

Biomass content: the biomass to be calculated may include cellulose, hemicelluloses and lignin. Exemplary methods for determining ligno-cellulosic content, total carbohydrate content and/or cellulose and hemicellulose content are provided in the Examples section herein below.

DNA content: may be determined, for example, using spectrophotometry analysis at 260 nm.

In some embodiments, the pellet is characterized by a protein:biomass ratio in the range of about 3:1-1:5 (w/w).

In some embodiments, the pellet is characterized by a DNA:biomass ratio in the range of about 1:400-1:25 (w/w), for example about 1:300-1:100.

The pellet preparation of the present invention has a cellulolytic activity, i.e., it is capable of hydrolyzing cellulosic substrates. An exemplary assay for determining the ability of the preparation to degrade cellulosic substrates is provided in the Examples section hereinbelow.

The enzyme content of the pellet preparation includes a variety of enzymatic activities. In particular, the preparation comprises a variety of carbohydrate active enzymes. As used herein, the term “carbohydrate active enzyme” refers to an enzyme that catalyzes the breakdown or modification of carbohydrates and glycoconjugates. The broad group of carbohydrate active enzymes is divided into enzyme classes and further into enzyme families according to a standard classification system (Cantarel et al. 2009 Nucleic Acids Res 37:D233-238). According to this classification system, four enzyme classes are defined, namely glycoside hydrolases, glycosyl transferases, polysaccharide lyases and carbohydrate esterases. An informative and updated classification of carbohydrate active enzymes is available on the Carbohydrate-Active Enzymes (CAZy) server (www.cazy.org) and/or CAZypedia (www.cazypedia.org).

In some embodiments, the pellet preparation comprises at least one endocellulase and at least one exocellulase.

In some embodiments, the pellet preparation comprises at least one hemicellulase. In some embodiments, the hemicellulase is selected from the group consisting of a xylanase, and arabinofuranosidase, an acetyl xylan esterase, a glucuronidase, an endo-galactanase, a mannanase, an endo-arabinase, an exo-arabinase, an exo-galactanase, a ferulic acid esterase, a galactomannanase, a xylogluconase, and mixtures thereof.

Beta-Glucosidase

In some embodiments, the compositions of the present invention comprise a beta-glucosidase. As used herein, the term “β-glucosidase” refers to an enzyme that hydrolyzes terminal, non-reducing β-D-glucose residues from cello-oligodextrins. In particular, this type of enzyme cleaves cellobiose to generate two molecules of glucose.

In some embodiments, the beta-glucosidase originates from the cellulosome-producing microorganism, meaning that it is endogenously produced by the microorganism. The beta-glucosidase, according to some embodiments, is a recombinant enzyme that is expressed and secreted by the microorganism, which has been genetically modified to produce it. In some embodiments, the beta-glucosidase is incorporated into the cellulosome of the microorganism. Incorporation of the enzyme into the cellulosome can be achieved, for example, via a dockerin present in the beta-glucosidase and a matching cohesin present in a scaffoldin subunit of the cellulosome, or vice versa, namely, via a cohesin present in the beta-glucosidase and a matching dockerin present in a scaffoldin subunit (type-II interaction). In additional embodiments, the beta-glucosidase comprises a cellulose-binding module.

The beta-glucosidase according to these embodiments is extra-cellular and detectable, meaning that it is secreted from the microorganism cells and it is possible to detect its activity after collection of the pellet. Detection of beta-glucosidase activity is typically performed using p-Nitrophenyl-beta-D-glucoside (pNPG) or cellobiose as a substrate by assays well known in the art.

In other embodiments, the beta-glucosidase is exogenous, meaning that it is added to the composition exogenously rather than produced by the microorganism. The exogenously added beta-glucosidase may be a commercially available beta-glucosidase, available, for example, from Codexis Inc. Alternatively, it may be prepared by means of well known recombinant methods, or by classical purification methods. Sources of beta-glucosidase suitable for use in accordance with the present invention include bacterial and fungal sources. In some embodiments, an exogenous beta-glucosidase is added to a composition which includes an unprocessed pellet of a microorganism that naturally produces a beta-glucosidase as a native enzyme. For example, it may be added to a composition containing an unprocessed pellet of a microorganism that produces an intracellular beta-glucosidase (which is undetectable in the pellet preparation).

In some embodiments, the exogenously added beta-glucosidase is a thermostable beta-glucosidase. In some embodiments, the beta-glucosidase is thermostable in temperatures above 50° C., above 60° C., for example above 70° C., in the range of 60-80° C. Each possibility represents a separate embodiment of the invention.

In some embodiments, the exogenously added beta-glucosidase is characterized by an optimum temperature for activity above 50° C., for example above 60° C., above 70° C., in the range of 60-80° C. Each possibility represents a separate embodiment of the invention.

In some embodiments, the beta-glucosidase originates from a thermophilic microorganism. In the case of a beta-glucosidase that is obtained from a thermophilic microorganism, the optimum temperature for the enzyme is usually the optimal growth temperature of the microorganism.

Examples of suitable sources of thermostable beta-glucosidases in the temperature optimum range of 60-80° C. include: Agrobacterium tumefaciens, Aspergillus aculeatus, Aspergillus foetidus, Aspergillus fumigatus, Aspergillus japonicas, Aspergillus niger, Aspergillus oryzae, Aspergillus pulverulentus, Aspergillus pulverulentus YM-80, Aspergillus tubingensis, Aspergillus wentii, Athelia rolfsi, Aureobasidium pullulans, Caldicellulosiruptor saccharolyticus, Cellvibrio gilvus Cellulomonas biazotea, Ceriporiopsis subvennispora, Ceriporiopsis subvermispora CS-1, Chaetomium thermophilum, Citrus sinensis, Clostridium stercorariurn, Clostridium thermocellum, Evernia prunastri, Fomitopsis palustris, Fusarium oxysporum, Hemicarpenteles ornatus, Hordeuin vulgare, Humicola sp., Hypocrea jecorina, Lenzites trabea, Melanocamus sp., Melanocarpus sp. MTCC 3922, Millerozyma farinosa, Myceliophthora heterothallica, Paecilomyces sp. J18, Penicillium aurantiogriseum, Penicillium brasilianum, Penicillium brasilianum IBT 20888, Penicillium decumbens, Penicillium purpurogenum, Penicillium purpurogenum KJS506, Penicillium verruculosum, Periconia sp., Phoma sp., Physarum polycephalum, Pyrococcus furiosus, Rhizomucor miehei, Sclerotinia sclerotiorum, Sulfolobus solfinataricus, Talaromyces emersonii, Talaromyces emersonii CBS 814.70, Termitomyces clypeatus, Thermoascus aurantiacus, Thermobispora bispora, Thermomyces lanuginosus, Themotoga maritime and Thermus thermophiles (found in BRENDA database, available at: www.brenda-enzymes.org). Each possibility represents a separate embodiment of the invention.

In some embodiments, the beta-glucosidase is from a bacterial source. In other embodiments, the beta-glucosidase is from a fungal source.

Particular, non-limiting, examples include Clostridium thermnocellum, e.g. BglA (accession number CAA42814), and Thermoanaerobacter brockii, e.g., CglT (accession number CAA91220).

In some embodiments, the amount of the exogenously added beta-glucosidase in the composition is in the range of about 0.001-10% (w/w) or any amount therebetween, for example about 0.01-5% (w/w) or any amount therebetween. Each possibility represents a separate embodiment of the invention.

In some embodiments, the β-glucosidase is classified in a glycoside hydrolase family selected from the group consisting of family 1, 3, 9, 30 and 116. Each possibility represents a separate embodiment of the invention.

Cellulose Hydrolysis by the Preparation:

The compositions of the present invention may be utilized for hydrolysis of cellulosic substrates, including ligno-cellulosic substrates.

By the term “cellulosic substrate”, it is meant any substrate that contains cellulose, in particular substrates derived from plant sources that contain cellulose, examples of which were provided hereinabove. The term “cellulosic substrate” further encompasses hemicellulose-containing substrates and ligno-cellulosic substrates.

In some embodiment, the concentration of the cellulosic substrate in the reaction mixture is about 5% or more, for example about 10% or more, in the range of about 5-30% or any amount therebetween, for example about 10-20% or any amount therebetween. Each possibility represents a separate embodiment of the invention.

In some embodiments, the weight ratio between the cellulolytic composition of the present invention and the cellulosic substrate is in the range of about 2-20% or any ratio therebetween, for example about 5-15% or any ratio therebetween. Each possibility represents a separate embodiment of the invention.

The reaction mixture may be incubated for from about 4 hours to about 120 hours, or any amount therebetween, for example between about 20-100 hours or any amount therebetween, at a temperature from about 30° C. to about 80° C., preferably above 60° C., or any temperature therebetween. Each possibility represents a separate embodiment of the invention. The optimal pH and temperature depend on the microorganism used for the production of the enzyme preparation, as well as the type of beta-glucosidase in the composition (if added).

By relieving end-product inhibition of endoxylanases and exo/endo-glucanases (such as xylobiose and cellobiose), it may be possible to further enhance the hydrolysis of the cellulosic material.

Following incubation, the reaction products can be used for further processing, for example as a substrate for producing ethanol, butanol, sugar alcohols, lactic acid, acetic acid, production of fuels, e.g., biofuels such as synthetic liquids or gases, such as syngas, or the end products may be concentrated and purified using standard methods as known in the art.

The degradation products typically comprise mono-, di- and oligosaccharide, including but not limited to glucose, xylose, cellobiose, xylobiose, ellotriose, cellotetraose, arabinose, and/or xylotriose.

Recycling the Enzymatic Complex:

Recycling of the enzymatic preparation refers to the repeated use of the same enzymatic preparation for successive hydrolyses of cellulosic substrates. Recycling typically comprises contacting a first sample of a cellulosic substrate with the enzyme composition to obtain hydrolysis of the first sample of a cellulosic substrate into hydrolysis products; (ii) collecting the hydrolysis products; and (iii) contacting a second sample of a cellulosic substrate with the enzyme composition to obtain hydrolysis of the second sample of a cellulosic substrate into hydrolysis products.

The ability to recycle the enzymes for successive fermentations can substantially reduce process costs. Advantageously, the compositions of the present invention can be simply recycled by subjecting samples to brief centrifugation (or other means of separation such as filtration through a micro sieve membrane) and replacing the supernatant fluids with fresh cellulosic substrate.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

Materials and Methods:

Biomass pretreatment: The biomass used in the experiments described below was subjected to one of the following pretreatments:

“Alkali treatment”—dry or wet-milled biomass was treated with 1-3% of NaOH at 120° C. for 15 minutes;

“Mercerize treatment”—dry or wet milled alkali biomass, hard wood paper or soft wood paper, or micro crystalline cellulose was incubated with 15-40% NaOH for 1-5 hours in room temperature, after which the slurry was naturalized with 4% sulfuric acid;

“Amorphous treatment”—dry biomass was incubated in water for several minutes, after which the slurry was mixed with 83% sulfuric acid until it became a gel. Next, 5 volumes of water were added to the slurry, and the mixture was then mixed for 30 minutes, centrifuged for 5 minutes, washed and naturalized by adding solid NaCO₃.

Preparation of enzymatically active pellets: A 1.3 L bioreactor was used with 1.2 L medium containing 0.5% to 3% dry biomass together with 0.5 g/L MgCl₂, 1.3 g/L (NH₄)₂SO₄, 1.33 g/L KH₂PO₄, 4.39 g/L K₂HPO₄, 5 g/L yeast extract, 2 mg/L resazurin, 0.5 ml/l antifoam, 1.25 mg/L FeSO₄, 73.5 mg/L CaCl₂, 1.5 g/L apple pectin. Medium was autoclaved for 15 minutes at 121° C. and purged with nitrogen and 1.3 g/L cysteine-HCl. The fermentation was done at 60° C., at constant pH of 7.2 for 24 to 72 hours, or until NaOH was no longer consumed. Samples of medium were centrifuged for 10 minutes at 17,000 g. The pellet was used as is for hydrolysis tests.

Hydrolysis assay: Activity assays of pelleted cells were done on 10% MCC in 50 mM citrate buffer, pH 6.0; 20 mM CaCl₂, 1 mM of DTT, 0.5% -2% of surfactant as PEG 2000 or Tween 80, and 0.03 to 0.3 mg/ml of the beta-glucosidase CglT from T. brockii or BglA from C. thermocellum, which were cloned into a pET28 vector and expressed in E. coli with a X6-His tag at the C-terminus. The incubation was done at 70° C. for 20 to 72 hours. The amount of reducing sugars (glucose equivalents) released by the enzyme was determined colorimetrically using 3,5-dinitrosalicylic acid (DNS) reagent and calculated based on a glucose calibration curve.

Protein content: Determined using the BCA protein assay with bovine serum albumin as standard.

### Example 1

### Production of Cell-Associated Cellulosomal Pellet by Clostridium thermocellum and Clostridium clariflavum

Clostridium thermocellum (Ct) DSM 1313 and Clostridium clariflavum (Cc), both are anaerobic cellulosome-producing bacteria, were first grown in a serum bottle containing 100 ml of 0.8% cellobiose, 0.5 g/L MgCl₂, 1.3 g/L (NH₄)₂SO₄, 0.5 g/L KH₂PO₄, 0.5 g/L K₂HPO₄, 10.5 g/l MOPS, 5 g/L yeast extract and 2 mg/L resazurin. The pH was adjusted to 7.2 with 10 M NaOH. Prior to the addition of the bacteria, the medium was boiled and 1.3 g/L cysteine-HCl was added and purged with nitrogen. Medium was autoclaved for 15 minutes at 121° C. The fermentation was done at 60° C., 16-24 h until the OD 595 reached 0.8 to 1.3. The obtained starter medium was then inoculated to a bioreactor containing 1.2 L comprising of: 1% mercerized MCC and containing 0.5 g/L MgCl₂, 1.3 g/L (NH₄)₂SO₄, 1.33 g/L KH₂PO₄, 4.39 g/L K₂HPO₄, 5 g/L yeast extract, 2 mg/L resazurin, 0.4% antifoam, 1.25 mg/L FeSO₄, 73.5 mg/L CaCl₂ and 1.5 g/L apple pectin. Medium was autoclaved for 15 minutes at 121° C. and purged with nitrogen before adding 1.3 g/L cysteine-HCl. The fermentation was done at 60° C., at constant pH of 7.2 for 24 h to 48 h. After the fermentation, the medium was centrifuged for 20 minutes at 3220 g.

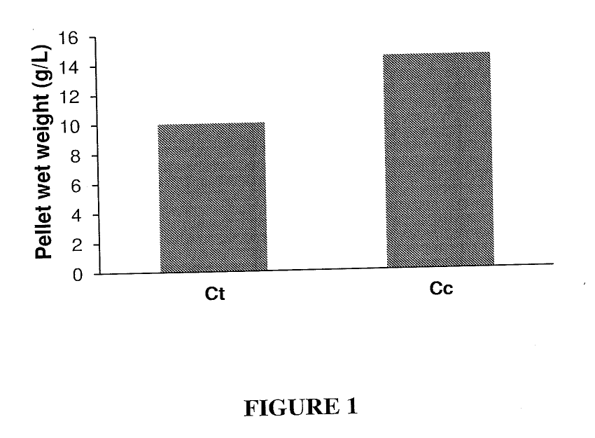

A 1 L sample from each culture was centrifuged, and the resulting pellet was weighted. The weights of the wet pellets are shown in FIG. 1.

### Example 2

### Production of Cell-Associated Cellulosomal Pellet on Different Biomass Types

C. thermocellum DSM 1313 was first grown in a serum bottle containing 100 ml of 0.8% cellobiose, 0.5 g/L MgCl₂, 1.3 g/L (NH₄)₂SO₄, 0.5 g/L KH₂PO₄, 0.5 K₂HPO₄, 10.5 g/l MOPS, 5 g/L yeast extract and 2 mg/L resazurin. The pH was adjusted to 7.2 with 10 M NaOH. Prior to the addition of the bacteria, the medium was boiled and 1.3 g/L cysteine-HCl was added and purged with nitrogen. The medium was autoclaved for 15 minutes at 121° C. The fermentation was done at 60° C. for 16 h to 24 h until the OD 595 reached 0.8 to 1.3. The obtained starter was used to inoculate 1.2 L media containing one of the following biomass types: 1% untreated switchgrass (“1% NA10 SG”), 2% untreated microcrystalline cellulose (“2% MCC”), 1% MCC, amorphous pretreatment (“1% amorphous MCC”), 1% MCC, mercerize pretreatment (“1% mercerize MCC”), 1% softwood, amorphous pretreatment (“1% amorphous softwood”), 0.5% wheat straw, mercerize and alkali pretreatment (“0.5% mercerize alkali wheat”), and 0.5% wheat straw, mercerize, acid and alkali pretreatment (“0.5% mercerize acid alkali wheat”). The media further included 0.5 g/L MgCl₂, 1.3 g/L (NH₄)₂SO₄, 1.33 g/L KH₂PO₄, 4.39 g/L K₂HPO₄, 5 g/L yeast extract, 2 mg/L resazurin, 0.05% antifoam, 1.25 mg/L FeSO₄, 73.5 mg/L CaC₂, 1.5 g/L apple pectin. Medium was autoclaved for 15 minutes at 121° C. and purged with nitrogen before adding 1.3 g/L cysteine-HCl. The fermentation was done at 60° C., at constant pH of 7.2 for 24-48 h, after which the pellet was centrifuged for 20 minutes at 3220 g.

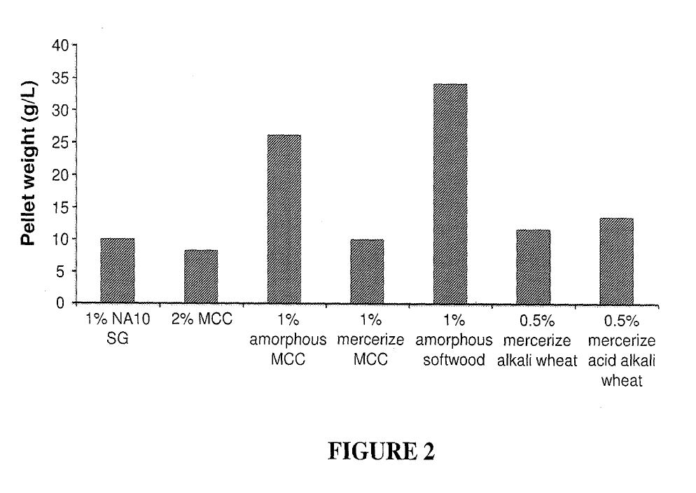

The wet-weight of 1 L samples was determined upon centrifugation. The results are shown in FIG. 2.

### Example 3

### Cellulose Hydrolysis by Cell-Associated Cellulosomal Pellet Produced Using Different Biomass

C. thermocellum was grown as described above on 0.8% cellobiose, 1% MCC or 0.5% mercerize and alkali pretreated wheat straw. A 1 L sample from each culture was centrifuged and the resulting pellets were weighted. The pellet weight was 4 g/l for 0.8% cellobiose containing media, 8.2 g/l for 1% MCC and 9.7 g/l for 0.5% alkali mercerized wheat straw.

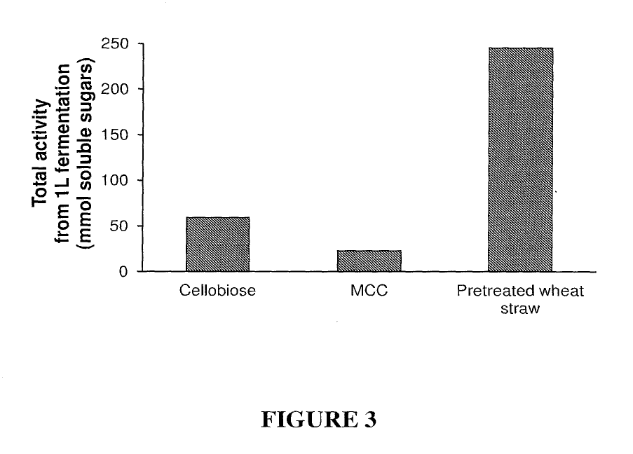

The resulting pellets were tested for cellulolytic activity as described above. FIG. 3 shows hydrolysis of 10% MCC after 20 hours at 70° C. with cell-associated cellulosomal pellet produced using the different biomass. The results are expressed as the total amount (mmol) of soluble reducing sugars measured at the end of the assay.

The amount of soluble sugars was determined using DNS method (G. L. Miller, Anal. Biochem. 1959, 31, 426). As can be seen in the figure, higher cellulolytic activity was observed when cells were grown on pre-treated natural plant material compared to previously demonstrated avicel or cellobiose.

### Example 4

### Composition of Cell-Associated Cellulosomal Pellet

Determination of the chemical compositions of cell-associated cellulosomal pellets obtained from C. thermocellum grown on different types of biomass and pretreatments was performed as follows:
1. Solid content (SC): 10±0.0001 g of wet initial cell-associated cellulosomal pellet (“pellet preparation”) sample (Pₒ) was weighted on analytical scale, dried at 105° C. up to constant weight, put in a desiccator containing silica gel, cooled up to room temperature and weighed (P_{d}).
   The SC of a wet pellet was calculated as:

SC, %=100(P_{d}/Pₒ)
2. Content of biomass fraction (BF): 3±0.0001 g of dried initial pellet preparation sample (Pₒ) obtained in Step 1 was extracted in 100 mL lab glass with 50 mL of boiling 0.25% NaOH for 1 h to remove proteins and salts. After cooling up to 40-50° C., contents were poured out into 50 mL PP tube and centrifuged at 3220 g for 10 min. The liquid phase was removed, and sediment was washed with 0.1% acetic acid, distillated water to pH 7 and with ethanol, removing the liquid phase at each washing by centrifugation. The wet residue was dried overnight at 60° C. and then at 105° C. up to constant weight, put in a desiccator containing silica gel, cooled up to room temperature and weighed (P_{d}).
   Content of BF in dry initial pellet preparation was calculated as:

BF_{d}, %=100(P_{d}/Pₒ)
Content of BF in wet initial pellet preparation was calculated as:

BF_{w}, %=SC(P_{d}/Pₒ)
Content of alkali soluble fraction (ASF) containing mainly proteins, in dry initial pellet preparation was calculated as:

ASF_{d}, %=100 [1−(P_{d}/Pₒ)]
Content of ASF in wet initial pellet preparation was calculated as:

ASF_{w}, %=100 [1−(P_{d}/Pₒ)]
3. Content of total carbohydrates (TH): 1±0.0001 g of dried biomass sample isolated after Step 2 (Pₒ) was extracted in 100 mL lab glass with 50 mL of boiling 0.5% sodium chlorite containing 0.5 ml of acetic acid for 1 h to remove lignin fraction. After cooling up to 40-50° C., contents were poured out into 50 mL PP tube and centrifuged at 3220 g for 10 min. The liquid phase was removed, and sediment was washed with distillated water to pH 7 and then with ethanol, removing the liquid phase at each washing by centrifugation. The wet residue containing TH was dried overnight at 60° C. and then at 105° C. up to constant weight, put in a desiccator containing silica gel, cooled up to room temperature and weighed (P_{d}).
   Content of TH in dry biomass (BM) was calculated as:

TH_{d}(BM), %=100(P_{d}/Pₒ)
Content of TH in dry initial pellet preparation (IP) was calculated as:

TH_{d}(IP), %=BF_{d}(P_{d}/Pₒ)
Content of TH in wet initial pellet preparation (IP) was calculated as:

TH_{w}(IP), %=0.01 SC×BF_{d}(P_{d}/Pₒ)
4. Content of Cellulose (C) & Hemicelluloses (H): 0.5±0.0001 g of dry TH isolated after Step 3 (Pₒ) was extracted in 100 mL lab glass with 50 ml of boiling 2% hydrochloric acid for 2 h to remove H-fraction. After cooling up to 40-50° C., contents were poured out into 50 mL PP tube and centrifuged at 3220 g for 10 min. The liquid phase was removed, and sediment was washed with distillated water, 0.5% of sodium bicarbonate, distillated water to pH 7 and then with ethanol, removing the liquid phase at each washing by centrifugation. The wet residue containing cellulose fraction was dried overnight at 60° C. and then at 105° C. up to constant weight, put in a desiccator containing silica gel. cooled up to room temperature and weighed (P_{d}).
   Content of C-fraction in dry TH was found as:

C_{d}(TH), %=100(P_{d}/Pₒ)
Content of H-fraction in dry TH was found as:

C_{d}(TH), %=100 [1−(P_{d}/Pₒ)]

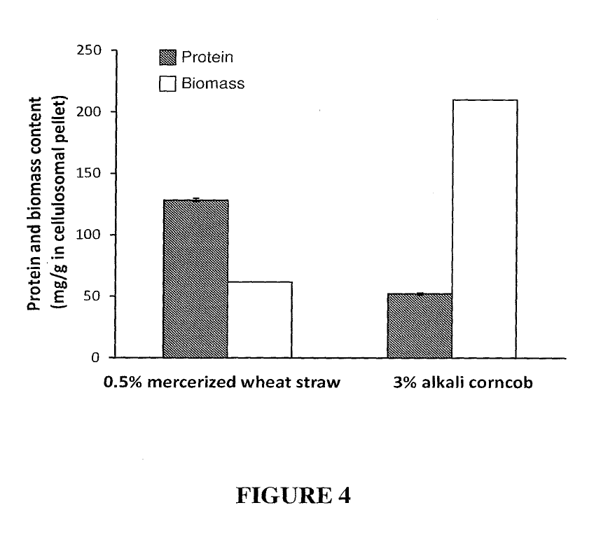

Protein content and total biomass content were determined for cell-associated cellulosomal pellets obtained from C. thermocellum grown on two different types of biomass and pretreatments. FIG. 4 shows the pellet composition (obtained from 1 L samples of fermentation broth). Black bars represent protein concentration and white bars represent biomass concentration.

Further analysis of the composition of cell-associated cellulosomal pellets obtained from C. thermocellum grown on 0.5% mercerized alkali wheat straw is detailed in the Table below.

The DNA content of the pellet preparations was in the range of 623-860 microgram/gram wet pellet.

Composition of dry preparation

| Percentage, % | Ingredient |
|---|---|
| 61 ± 1 | Alkali soluble fraction |
| 50 ± 7.1 | * Protein |
| 14.0 ± 0.2 | Lignin |
| 25.0 ± 0.2 | Total carbohydrates |
| * 22.3 ± 0.2 | * Cellulose |
| * 2.7 ± 0.2 | * Hemicelluloses |

### Example 5

### Cellulose Hydrolysis by Cell-Associated Cellulosomal Pellet in Comparison to a Commercial Enzyme Cocktail

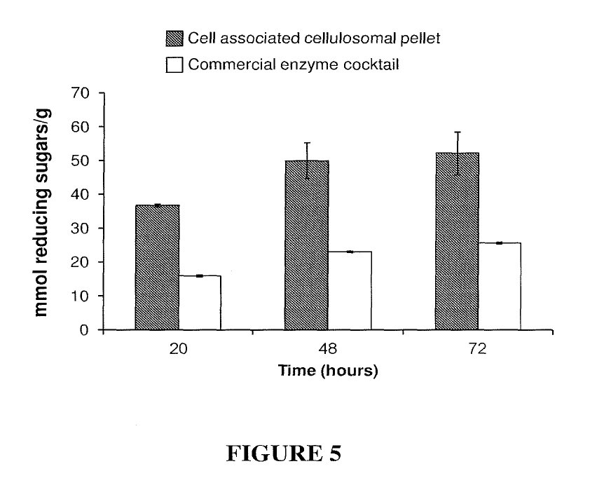

Cellulolytic activity of pellets obtained from C. thermocellum grown on alkali mercerize pretreated wheat straw and supplemented with beta-glucosidase was compared to that of the commercial enzyme cocktail Accellerase® 1500 (Genencor). FIG. 5 shows hydrolysis of 10% MCC by cell-associated cellulosomal pellet (with exogenously added beta glucosidase) and the commercial enzyme cocktail in 10% w/w enzyme/substrate ratios at 70° C. and 50° C. respectively. The activity is expressed as mmol reducing sugars per gram pellet/enzyme cocktail. Black bars represent cell-associated cellulosomal pellet, white bars represents the commercial cocktail.

### Example 6

### Cellulose Hydrolysis by Cell-Associated Cellulosomal Pellet at Different Temperatures

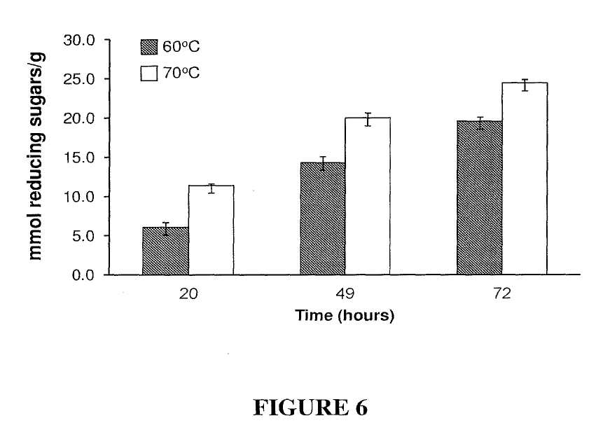

Activity on 10% MCC of cell-associated cellulosomal pellet (10% w/w) obtained from C. thermocellum grown on 0.5% alkali-acid-mercerize pretreated wheat straw and supplemented with beta-glucosidase was tested at 60° C. and 70° C. The results of the comparison are shown in FIG. 6, expressed as mmol reducing sugars per gram pellet. As can be seen in the figure, a significant improvement of hydrolysis was observed at the higher temperature, most likely as a result of providing the optimal activity temperature of the individual hydrolytic enzymes.

### Example 7

### Enhancing Degradation with Exogenous Beta-Glucosidase

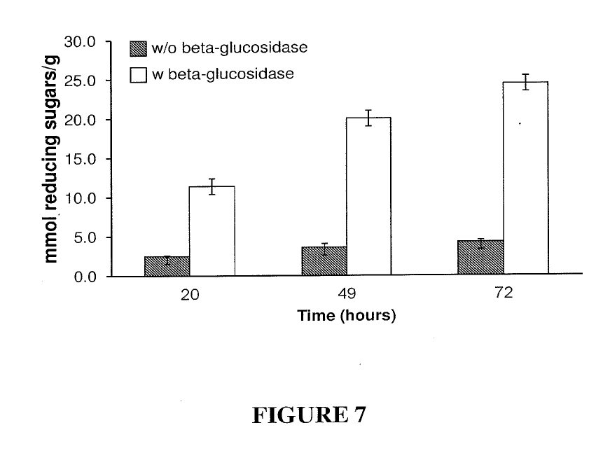

The influence of beta glucosidase addition on hydrolysis rate of 10% MCC by cell-associated cellulosomal pellet was tested by performing the hydrolysis assay with or without the addition of a beta glucosidase at a 0.032% w/w (at 70° C.). The results are shown in FIG. 7. Black bars represent cell-associated cellulosomal pellet. White bars represent cell-associated cellulosomal pellet with addition of beta glucosidase.

### Example 8

### Recycling the Enzymatic Complex

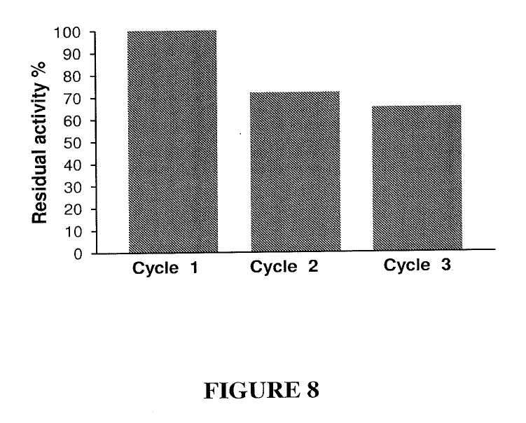

FIG. 8 demonstrates three consecutive recycling of the cell-associated cellulosomal pellet complex on a MCC. Three (3) rounds of enzyme recycling were done for 24 hours each at 70° C. Between each cycle, the samples were centrifuged briefly and the supernatant was replaced with fresh biomass. Over 65% of enzymatic activity was retained after the third cycle, demonstrating the feasibility of the method.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

What is claimed is:

1. A process for producing a cellulolytic enzyme preparation, the process comprising:
(i) culturing a cellulosome-producing microorganism in a medium comprising a cellulosic material until late exponential phase or stationery phase,
(ii) centrifuging or filtering the medium following said culturing to obtain a pellet;
(iii) separating the obtained pellet from the supernatant and inactivating the cells of the microorganism; and
(iv) using the obtained pellet without purification of enzymes or other components to enzymatically hydrolyze cellulosic substrates.

2. The process of claim 1, wherein the cellulosic material is selected from the group consisting of wheat straw, switchgrass, corn cob, corn stover, sorghum straw, cotton straw, bagasse, energy cane, hard wood paper, soft wood paper, and a combination thereof and/or derived from a plant selected from the group consisting of a Populous species, a Salix species, an Acacia species, a Tamarix species, Arundo donax, Miscanthus giganteus, and a combination thereof.

3. The process of claim 1, wherein the medium further comprises pectin.

4. The process of claim 1, further comprising adding a beta-glucosidase to the obtained pellet.

5. A method for hydrolyzing a cellulosic substrate, the method comprising producing the cellulolytic enzyme preparation according to claim 1, and contacting the cellulosic substrate with said cellulolytic enzyme preparation.

6. The method of claim 5, further comprising recycling the enzyme preparation for one or more successive hydrolyses.

7. A method for hydrolyzing a cellulosic substrate, the method comprising contacting the cellulosic substrate with an enzyme composition comprising:
an unprocessed pellet of a cellulosome-producing microorganism, wherein cells of the cellulosome-producing microorganism contained within the pellet are inactivated; and
detectable extra-cellular beta-glucosidase.

8. The method of claim 7, further comprising recycling the enzyme composition for one or more successive hydrolyses.
